(19) United States
(12) Patent Application Publication
(10) Pub. No.: US 2024/0285189 A1
(43) Pub. Date: Aug. 29, 2024

(54) FALL DETECTION

(71) Applicant: Essence SmartCare Ltd., Herzlia Pituach (IL)

(72) Inventors: Ohad AMIR, Herzlia (IL); Jonathan Mark SCHNAPP, Tel Aviv (IL)

(21) Appl. No.: 18/573,405

(22) PCT Filed: Jul. 21, 2022
(86) PCT No.: PCT/IL2022/050790

§ 371 Date: Dec. 21, 2023

(30) Foreign Application Priority Data
Jul. 22, 2021 (GB) 2110551.5

(51) Int. Cl. A61B 5/11; G01S 7/41

(52) U.S. Cl. CPC A61B 5/1117; G01S 7/415

(57) ABSTRACT

One embodiment relates to a computer implemented method for detecting if a person has fallen in an environment comprising multiple people. The method comprises controlling an active reflected wave detector to measure wave reflections from the multiple people to receive measured wave reflection data that is obtained by the active reflected wave detector. The method further comprises detecting if a person has fallen in the environment using at least a portion of the measured wave reflection data, wherein a fall status of a nearest person in the environment, being a person of the multiple people that is nearest to the active reflected wave detector, is deterministic of a detected fall.

### RELATED APPLICATIONS

This application claims the benefit of priority of Great Britain Patent Application No. 2110551.5 filed on 22 Jul. 2021, the contents of which are incorporated herein by reference in their entirety.

### TECHNICAL FIELD

The present invention relates generally to fall detection, in particular a device and method for detecting if a person has fallen in an environment.

### BACKGROUND

There is a need to use a monitoring system to automatically detect when a person has fallen in a designated space, for example in an interior of a building. For example, an elderly person may end up in a hazardous situation when they have fallen and are unable to call for help, or unable to do so quickly.

Some known systems have been developed in which the person wears a pendant which has an accelerometer in it to detect a fall based on kinematics. The pendant upon detecting a fall can transmit an alert signal. However the person may not want to wear, or may be in any case not wearing, the pendant.

Other reflected-wave based systems such as radar (whether radio wave, microwave or millimeter wave), lidar or sonar, are known to monitor a person in a designated space.

### SUMMARY

The inventors have identified that when using an active reflected wave detector to detect a target object (e.g. human), due to the potential for multi-path reflections, in addition to receiving wave reflections involving waves that have travelled from a direct path between the detector and the target object, the active reflected wave detector may also receive wave reflections involving waves that have travelled on an indirect path between the detector and the target object. Such indirect reflection paths leads to detections from which it may be falsely concluded that there is an object at a certain position, so the object attributed as being at that certain position may be considered a phantom object or, in other words, an echo of the actual object.

When performing fall detection based on measured wave reflection data, the presence of phantom targets has potential to decrease the accuracy of the fall detection. For example, the fall detector may conclude that a person has fallen when, unbeknownst to the fall detector, the alleged fallen person is a phantom object.

In the event of a processor detecting multiple objects in an environment based on processing measured wave reflection data obtained by an active reflected wave detector, this may be caused by multiple people being present in the environment or a single person and multi-path reflections.

The present inventors have recognised, however, that should there be an actual object and one or more corresponding phantom objects represented in the measured wave reflection data, the actual object is always the closest of these objects to the active reflected wave detector. In a scenario in which multiple actual objects are present, since the detector may not be able to distinguish between this scenario and one in which there is a single actual object and one or more phantom objects, the present invention utilizes a rationale that a fall detection does not correspond to a phantom object if the object for which the fall detection is made is for a nearest object.

On the other hand, if there are multiple objects appearing in the measured wave reflection data and the nearest object is determined not to be a fallen person, then the nearest object may be assumed to be an able-bodied person. In some embodiments, the fall status of a non-nearest object is ignored on the basis that either the non-nearest object is a phantom object or, if the non-nearest object is an actual other person that has fallen, then the able-bodied person can assist them.

According to one aspect of the present disclosure there is provided a computer implemented method for detecting if a person has fallen in an environment comprising multiple people, the method comprising: controlling an active reflected wave detector to measure wave reflections from the multiple people to receive measured wave reflection data that is obtained by the active reflected wave detector; and detecting if a person has fallen in the environment using at least a portion of the measured wave reflection data, wherein a fall status of a nearest person in the environment, being a person of the multiple people that is nearest to the active reflected wave detector, is deterministic of a detected fall.

Preferably, a fall status of a non-nearest person in the environment, being a person of the multiple people that is not nearest to the active reflected wave detector, is not deterministic of a detected fall.

The method may comprise detecting that a person has fallen in the environment if the fall status of the nearest person is that they are in a fall state, and the fall status of a non-nearest person is that they are in a non-fall state.

The method may comprise detecting that a person has fallen in the environment if the fall status of the nearest person is that they are in a fall state, and the fall status of a non-nearest person is that they are in a fall state.

If the fall status of the nearest person is that they are in a non-fall state, the method may comprise detecting that a person has not fallen in the environment.

The method may further comprise: detecting a plurality of objects from the measured wave reflection data that is obtained by the active reflected wave detector; identifying wave reflection data associated with an object, of the plurality of objects, that is nearest to the active reflected wave detector and is identified as being human, and detecting if a person has fallen in the environment by determining the fall status using the wave reflection data associated with the object in the environment that is nearest to the active reflected wave detector.

The detecting if a person has fallen in the environment may comprise disregarding wave reflection data associated with any objects that are not nearest to the active reflected wave detector.

The plurality of objects may comprise the multiple people.

Different people may correspond to different objects of the plurality of objects.

The plurality of objects may further comprise one or more phantom objects.

The method may comprise supplying the wave reflection data, that is associated with the object in the environment that is nearest to the active reflected wave detector, to a classifier that has been trained with training data, the classifier configured so that the detecting if a person has fallen in the environment is based on the training data and the wave reflection data that is associated with the object in the environment that is nearest to the active reflected wave detector. The method may comprise detecting if a person has fallen in the environment using the measured wave reflection data from the multiple people, the method comprising supplying the wave reflection data from the multiple people to a classifier that has been trained with training data, the classifier configured so that the detecting if a person has fallen in the environment is based on the training data and the wave reflection data from the multiple people.

The training data may comprise one or more wave reflection data training sets associated with a multi-occupancy environment, each wave reflection data training set labelled as being representative that a person has fallen in the environment or being representative that a person has not fallen in the environment.

The training data may comprise at least one of
a first wave reflection data training set comprising wave reflection data of a nearest person to an active reflected wave detector being in a non-fall state, the first wave reflection data training set labelled as being representative that a person has not fallen in the environment;

a second wave reflection data training set comprising wave reflection data of a nearest person to an active reflected wave detector being in a fall state and wave reflection data of a non-nearest person to an active reflected wave detector being in a non-fall state, the second wave reflection data training set labelled as being representative that a person has fallen in the environment; and a third wave reflection data training set comprising wave reflection data of a nearest person to an active reflected wave detector being in a fall state and wave reflection data of a non-nearest person to an active reflected wave detector being in a fall state, the third wave reflection data training set labelled as being representative that a person has fallen in the environment.

The first wave reflection data training set may comprise:
wave reflection data of a nearest person to an active reflected wave detector being in a non-fall state and wave reflection data of a non-nearest person to an active reflected wave detector being in a non-fall state, being labelled as being representative that a person has not fallen in the environment; and
wave reflection data of a nearest person to an active reflected wave detector being in a non-fall state and wave reflection data of a non-nearest person to an active reflected wave detector being in a fall state, labelled as being representative that a person has not fallen in the environment.

The fall status of the nearest person may represent whether the nearest person is in a position representative of them having fallen, wherein the nearest person is in a fall state if the nearest person is in a position representative of them having fallen, and is in a non-fall state if the nearest person is not in a position representative of them having fallen.

The method may further comprise controlling the issuance of a fall detection alert in response to detecting that a person has fallen in the environment.

The controlling the active reflected wave detector to measure wave reflections from the environment may be performed in response to detecting motion in the environment based on receiving motion detection data from a motion detector.

The controlling the active reflected wave detector to measure wave reflections from the environment may be performed upon expiry of a time window that commences in response to the motion sensor detecting motion of a person.

The active reflected wave detector may be a radar sensor or a sonar sensor.

The method may comprise using a radial distance in 2-dimensional horizontal space to determine that the nearest person is nearest to the active reflected wave detector.

The method may comprise using a radial distance in 3-dimensional space to determine that the nearest person is nearest to the active reflected wave detector.

The method steps of one or more embodiments described herein may be performed by a single processor or on a distributed processing system. For example, the method steps of one or more embodiments described herein may be performed by a processing system that is distributed amongst a plurality of devices that include multiple devices geographically separately from each other.

According to another aspect of the present disclosure there is provided a system configured to perform any of the steps of the methods described herein. For example, a system may be provided for detecting if a person has fallen in an environment comprising multiple people, the system comprising a device for controlling an active reflected wave detector to measure wave reflections from the multiple people to receive measured wave reflection data that is obtained by the active reflected wave detector;
wherein the system is configured to detect if a person has fallen in the environment using at least a portion of the measured wave reflection data, wherein a fall status of a nearest person in the environment, being a person of the multiple people that is nearest to the active reflected wave detector, is deterministic of a detected fall.

The detecting if a person has fallen in the environment using at least a portion of the measured wave reflection data may be performed, at least in part, by at least one device that is remote from said device. Further, the detecting if a person has fallen in the environment using at least a portion of the measured wave reflection data may be performed, at least in part, by said device.

The system may further comprise the active reflected wave detector.

According to another aspect of the present disclosure there is provided a computer-readable storage medium comprising instructions which, when executed by a processor cause the processor to perform the method steps of one or more embodiments described herein.

The instructions may be provided on one or more carriers. For example there may be one or more non-transient memories, e.g. a EEPROM (e.g. a flash memory) a disk, CD- or DVD-ROM, programmed memory such as read-only memory (e.g. for Firmware), one or more transient memories (e.g. RAM), and/or a data carrier(s) such as an optical or electrical signal carrier. The memory/memories may be integrated into a corresponding processing chip and/or separate to the chip. Code (and/or data) to implement embodiments of the present disclosure may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language.

According to another aspect of the present disclosure there is provided a device for detecting if a person has fallen in an environment comprising multiple people, the device comprising: a processor, wherein the processor is configured to: control an active reflected wave detector to measure wave reflections from the multiple people to receive measured wave reflection data that is obtained by the active reflected wave detector; and detect if a person has fallen in the environment using at least a portion of the measured wave reflection data, wherein a fall status of a nearest person in the environment, being a person of the multiple people that is nearest to the active reflected wave detector, is deterministic of a detected fall.

The processor may be configured to perform the method steps of one or more embodiments described herein.

The device may further comprise the active reflected wave detector.

The processor may be further configured to:
control an active reflected wave detector to measure wave reflections from an environment comprising a single person to receive measured wave reflection data that is obtained by the active reflected wave detector;
detect a plurality of objects from the measured wave reflection data that is obtained by the active reflected wave detector, the plurality of objects comprising an object corresponding to the single person and one or more phantom objects;
identify wave reflection data associated with an object, of the plurality of objects, that is nearest to the active reflected wave detector, and detect if a person has fallen in the environment by determining the fall status using the wave reflection data associated with the object in the environment that is nearest to the active reflected wave detector.

The processor may be configured to disregard wave reflection data associated with any objects that are not nearest to the active reflected wave detector in said detection of a person has fallen in the environment.

From the perspective of the device, it does not implement two different processes depending on whether there is one actual person in the environment and one or more phantom objects, as compared with there being multiple actual people in the environment. That is, regardless of whether there is (i) a single person and one or more phantoms in the environment or (ii) multiple people in the environment, the operation of the processor is the same, as all non-nearest objects have no influence on the fall detection result, regardless of whether the non-nearest object is a real or a phantom object.

For example, the processor may be configured to:
detect a plurality of objects from the measured wave reflection data that is obtained by the active reflected wave detector to detect a plurality of objects;
identify wave reflection data associated with an object, of the plurality of objects, that is nearest to the active reflected wave detector and is identified as being human, and detect if a person has fallen in the environment by determining the fall status using the wave reflection data associated with the object in the environment that is nearest to the active reflected wave detector.

When multiple people are present in the environment the plurality of detected objects will correspond to the different people in the environment and may also include one or more phantom objects. In contrast, when a single person is present in the environment the plurality of detected objects will correspond to the single person and one or more phantom objects.

Thus according to another aspect of the present disclosure there is provided a computer implemented method for detecting if a person has fallen in an environment comprising at least one person, the method comprising:
controlling an active reflected wave detector to measure wave reflections from the environment to receive measured wave reflection data that is obtained by the active reflected wave detector; detecting a plurality of objects from the measured wave reflection data that is obtained by the active reflected wave detector;
identifying wave reflection data associated with an object, of the plurality of objects, that is nearest to the active reflected wave detector and is identified as being human, and
detecting if a person has fallen in the environment by determining the fall status using the wave reflection data associated with the object in the environment that is nearest to the active reflected wave detector.

According to another aspect of the present disclosure there is provided a non-transitory computer-readable storage medium comprising instructions for detecting if a person has fallen in an environment comprising at least one person, the instructions when executed by a processor cause the processor to:
control an active reflected wave detector to measure wave reflections from the environment to receive measured wave reflection data that is obtained by the active reflected wave detector; detect a plurality of objects from the measured wave reflection data that is obtained by the active reflected wave detector;
identifying wave reflection data associated with an object, of the plurality of objects, that is nearest to the active reflected wave detector and is identified as being human, and detect if a person has fallen in the environment by determining the fall status using the wave reflection data associated with the object in the environment that is nearest to the active reflected wave detector.

According to another aspect of the present disclosure there is provided a device for detecting if a person has fallen in an environment comprising at least one person, the device comprising: a processor, wherein the processor is configured to:
control an active reflected wave detector to measure wave reflections from the environment to receive measured wave reflection data that is obtained by the active reflected wave detector; detect a plurality of objects from the measured wave reflection data that is obtained by the active reflected wave detector;
identifying wave reflection data associated with an object, of the plurality of objects, that is nearest to the active reflected wave detector and is identified as being human, and
detect if a person has fallen in the environment by determining the fall status using the wave reflection data associated with the object in the environment that is nearest to the active reflected wave detector.

These and other aspects will be apparent from the embodiments described in the following. The scope of the present disclosure is not intended to be limited by this summary nor to implementations that necessarily solve any or all of the disadvantages noted.

### BRIEF DESCRIPTION OF THE SEVERAL VIEWS OF THE DRAWINGS

For a better understanding of the present disclosure and to show how embodiments may be put into effect, reference is made to the accompanying drawings in which:

FIG. 1a illustrates a 2-dimensional representation of an environment in which a device has been positioned;

FIG. 1b illustrates a 3-dimensional representation of the environment in which the device has been positioned;

FIG. 2 is a schematic block diagram of the device;

FIGS. 3a and 3b illustrates a human body with indications of reflections measured by a reflective wave detector when the person is in a standing non-fall state and in a fall state;

FIG. 4 illustrates a general process for detecting if a person has fallen in an environment comprising multiple people;

FIG. 5 illustrates a flowchart of an example implementation of the general process shown in FIG. 4; and

FIG. 6 illustrates a flowchart of a further example implementation of the general process shown in FIG. 4.

### DETAILED DESCRIPTION

In the following detailed description, reference is made to the accompanying drawings that form a part hereof, and in which is shown by way of illustration specific embodiments in which the inventive subject matter may be practiced. These embodiments are described in sufficient detail to enable those skilled in the art to practice them, and it is to be understood that other embodiments may be utilized, and that structural, logical, and electrical changes may be made without departing from the scope of the inventive subject matter. Such embodiments of the inventive subject matter may be referred to, individually and/or collectively, herein by the term “invention” merely for convenience and without intending to voluntarily limit the scope of this application to any single invention or inventive concept if more than one is in fact disclosed.

The following description is, therefore, not to be taken in a limited sense, and the scope of the inventive subject matter is defined by the appended claims and their equivalents.

In the following embodiments, like components are labelled with like reference numerals.

In the following embodiments, the term data store or memory is intended to encompass any computer readable storage medium and/or device (or collection of data storage mediums and/or devices). Examples of data stores include, but are not limited to, optical disks (e.g., CD-ROM, DVD-ROM, etc.), magnetic disks (e.g., hard disks, floppy disks, etc.), memory circuits (e.g., EEPROM, solid state drives, random-access memory (RAM), etc.), and/or the like.

As used herein, except wherein the context requires otherwise, the terms “comprises”, “includes”, “has” and grammatical variants of these terms, are not intended to be exhaustive. They are intended to allow for the possibility of further additives, components, integers or steps. The functions or algorithms described herein are implemented in hardware, software or a combination of software and hardware in one or more embodiments. The software comprises computer executable instructions stored on computer readable carrier media such as memory or other type of storage devices. Further, described functions may correspond to modules, which may be software, hardware, firmware, or any combination thereof. Multiple functions are performed in one or more modules as desired, and the embodiments described are merely examples. The software is executed on a digital signal processor, ASIC, microprocessor, or other type of processor.

Specific embodiments will now be described with reference to the drawings.

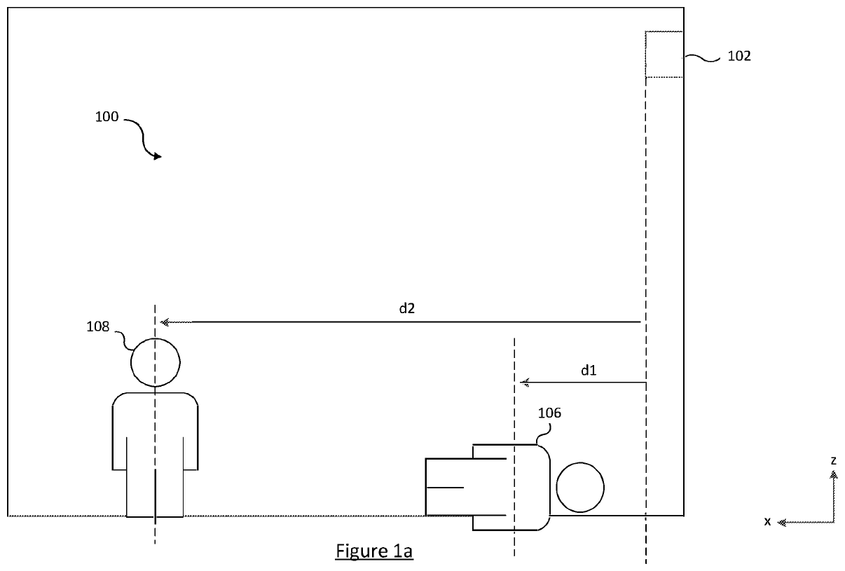

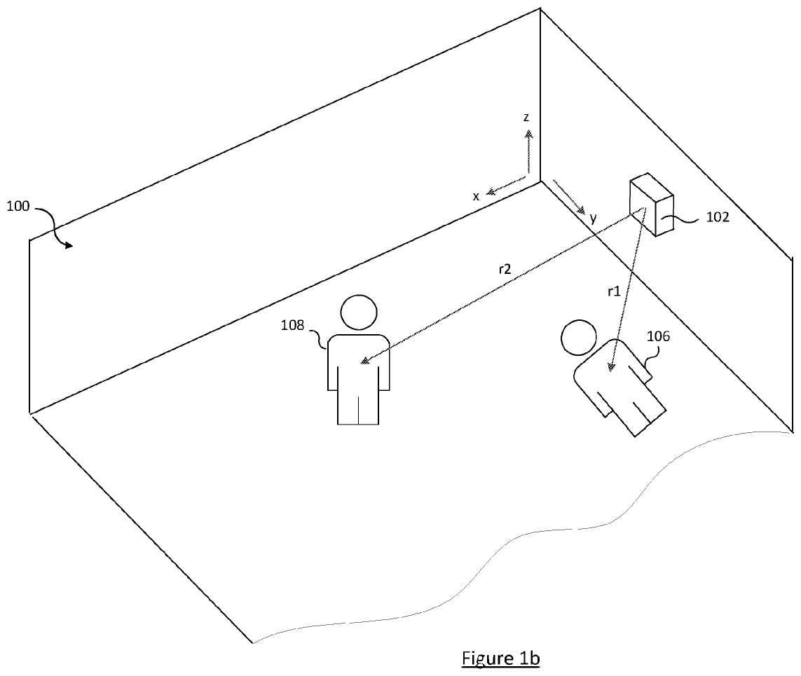

FIG. 1a illustrates a 2-dimensional representation of an environment 100 in which a device 102 (a fall detector device) has been positioned mounted to a wall. It will be appreciated that the device may also be mounted to a ceiling or on a post away from the ceiling and walls. The environment 100 may for example be an indoor space such as a room of a home, a nursing home, a public building or other indoor space. Alternatively the environment may be an outdoor space such as a courtyard or garden. The device 102 is configured to monitor the environment 100 in which a person or multiple persons may be present.

FIG. 1b illustrates a 3-dimensional representation of the environment 100 shown in FIG. 1a.

For illustration purposes only, FIGS. 1a and 1b shows the environment comprising two people, person 106 and person 108. It will be appreciated that the environment may comprise a different number of people than that shown in FIGS. 1 and 1b.

The present invention relates to the detection of a person 106 having fallen which is illustrated in FIGS. 1 and 1b. We refer herein to how close (i.e. how near) a person in the environment is to an active reflected wave detector of a device.

How close a person is to the active reflected wave detector can be measured by the device CPU in 1-dimensional space with reference to a horizontal direction, e.g. the x-direction shown in FIG. 1a. This may be used in some particular environments e.g. in a narrow hallway in which reflective wave measurement coordinates corresponding to places, that based on their location, can be concluded to be outside the hallway are ignored. However at least but not only in scenarios in which a person's location may be at a horizontal location defined by two dimensions, it is preferable to use a multi-dimensional distance measurement in order to most accurately determine the nearest object to the active reflected wave detector.

In this regard, how close a person is to the active reflected wave detector can be measured by the device CPU as a radial distance in 2-dimensional horizontal space. With reference to FIG. 1a, it can be seen that of the people in the environment 100, person 106 is nearest to the active reflected wave detector (in the example of FIG. 1a the active reflected wave detector is housed within the device 102). Expressed another way, the horizontal distance d1 (which is a radial distance in 2-dimensional horizontal space) between the active reflected wave detector and a position of a point taken as the location of the person 106 is shorter than the horizontal distance d2 (which is a radial distance in 2-dimensional horizontal space) between the active reflected wave detector and a position of a point taken as the location of the person 108.

It will be appreciated that the distances d1 and d2 may each be defined by two cartesian dimensions but for illustration purposes, the 2-dimensional horizontal space is shown with respect to a single dimension (the x-direction). The second dimension in the y direction is into or out of the page. As will be appreciated, the radial distance is the square root of the sum of the x-distance squared and the y-distance squared. In other embodiments, the closeness of a person to the active reflected wave detector may be measured in 3-dimensional space as a radial distance from the active reflected wave detector. As will be appreciated, the radial distance is the square root of the sum of the x-distance squared, the y-distance squared and the z-distance squared.

In a 3-dimensional spherical coordinate system, the position of a point may be taken as the location of a person is specified by the following three parameters: radial distance r, azimuthal angle θ, and polar angle φ. In the example of FIG. 1b, the the radial distance r1 between the active reflected wave detector and the person 106 is shorter than the radial distance r2 between the active reflected wave detector and the person 108. Measuring how close a person is to the active reflected wave detector as a radial distance in 2-dimensional horizontal space may provide similar performance to a radial distance in 3-dimensional space if the device 102 is mounted in the environment such that any fall detected is positioned in the environment lying in a plane that bisects or nearly bisects all positions in which a person may be located. This could be the case if the device 102 is for example at a height in the range of 0.5 to 1 meters (e.g. 0.75 m) above the floor.

As noted above in some arrangements of how the device is mounted in the environment, measuring how close a person is to the active reflected wave detector in 2-dimensional space may be sufficient to ensure that the closest object to the active reflected wave detector is not a phantom object. However, in some arrangements it is possible for the nearest object in 3-dimensional space to be a non-nearest object in 2-dimensional space, hence leading to an error in the fall detection by the device. For example, the device 102 (housing the active reflected wave detector) may be preferably mounted at least 1.8 m above the floor. Thus, it is preferable for the device CPU to measure how close a person is to the active reflected wave detector in 3-dimensional space to provide an accurate distance measurement regardless of the environment in which the device 102 is positioned and how the device 102 is mounted in the environment (e.g. the height of the active reflected wave detector above the floor).

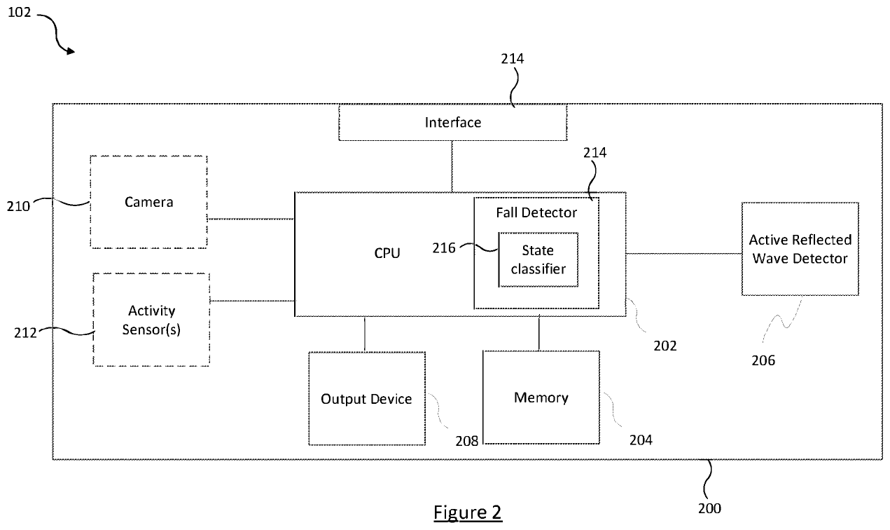

FIG. 2 illustrates a simplified view of the device 102. A shown in FIG. 2, the device 102 comprises a central processing unit (“CPU”) 202, to which is connected a memory 204. The functionality of the CPU 202 described herein may be implemented in code (software) stored on a memory (e.g. memory 204) comprising one or more storage media, and arranged for execution on a processor comprising one or more processing units. The storage media may be integrated into and/or separate from the CPU 202. The code is configured so as when fetched from the memory and executed on the processor to perform operations in line with embodiments discussed herein. Alternatively, it is not excluded that some or all of the functionality of the CPU 202 is implemented in dedicated hardware circuitry (e.g. ASIC(s), simple circuits, gates, logic, and/or configurable hardware circuitry like an FPGA. In other embodiments (not shown) a processing system executes the processing steps described herein, wherein the processing system may consist of the processor as described herein or may be comprised of distributed processing devices that may be distributed across two or more devices. Each processing device of the distributed processing devices may comprise any one or more of the processing devices or units referred to herein.

FIG. 2 shows the CPU 202 being connected to an active reflected wave detector 206.

The CPU 202 may optionally also be connected to a camera 210 and/or one or more activity sensor 212. While in the illustrated embodiment the activity sensor(s) 212, active reflected wave detector 206, and the camera 210 are separate from the CPU 202, in other embodiments, at least part of processing aspects of the activity sensor(s) 212 and/or active reflected wave detector 206 and/or camera 210 may be provided by a processor that also provides the CPU 202, and resources of the processor may be shared to provide the functions of the CPU 202 and the processing aspects of the activity sensor(s) 212 and/or active reflected wave detector 206 and/or camera 210. Similarly, functions of the CPU 202, such as those described herein, may be performed in the activity sensor(s) 212 and/or the active reflected wave detector 206 and/or the camera 210.

As shown in FIG. 2, a housing 200 of the device 102 may house the activity sensor(s) 212, the active reflected wave detector 206, and the camera 210. Alternatively, the activity sensor(s) 212 may be external to the device 102 and be coupled to the CPU 202 by way of a wired or wireless connection. Similarly, the active reflected wave detector 206 may be external to the device 102 and be coupled to the CPU 202 by way of a wired or wireless connection. Similarly, the camera 210 may be external to the device 102 and be coupled to the CPU 202 by way of a wired or wireless connection. Further, the outputs of the activity sensor(s) 212 and/or active reflected wave detector 206 and/or camera 210 may be wirelessly received from/via an intermediary device that relays, manipulates and/or in part produces their outputs.

The activity sensor(s) 212 are each configured to detect activity in the environment. In implementations that use multiple activity sensors, the multiple activity sensors may detect activity in different regions of the environment (e.g. different rooms of a home, or more preferably a different regions of the same room).

One example activity sensor 212 is a motion sensor. In implementations using a motion sensor 212, the CPU 202 is configured to detect motion in the environment based on an output of the motion sensor. The motion sensor may be a passive infrared (PIR) sensor. The motion sensor is preferably a PIR sensor, however it could be an active reflected wave sensor, for example radar, that detects motion based on the Doppler effect. For example, the motion sensor may be a radar based motion sensor which detects motion based on the Doppler component of a radar signal.

The activity sensor(s) 212 may include a microphone, a vibration sensor, and/or an infrared sensor. Other types of activity sensors are known to persons skilled in the art.

In an activated state, the active reflected wave detector 206 operates to measure wave reflections from the environment.

The active reflected wave detector 206 may operate in accordance with one of various reflected wave technologies. In operation, the CPU 202 uses the output of the active reflected wave detector 206 to determine the presence of a target object (e.g. human).

The active reflected wave detector 206 is a ranging detector. That is, in contrast with Doppler-only detectors, the active reflected wave detector 206 is configured to determine the location of an object (e.g. a person) in its field of view. This enables the CPU 202 to track the location of an object in the environment and also to determine which detected object is nearest.

In some implementations, the active reflected wave detector 206 may provide both a ranging based output and a Doppler-based output based on measuring wave reflections from the environment. In these implementations, the active reflected wave detector 206 is configured to detect motion in a region in the environment, and a dedicated motion sensor 212 is not required.

Preferably, the active reflected wave detector 206 is a radar sensor. The radar sensor 206 may use millimeter wave (mmWave) sensing technology. The radar is, in some embodiments, a continuous-wave radar, such as frequency modulated continuous wave (FMCW) technology.

Such a chip with such technology may be, for example, Texas Instruments Inc. part number iwr6843AOP. The radar may operate in microwave frequencies, e.g. in some embodiments a carrier wave in the range of 1-100 GHz (76-81 Ghz or 57-64 GHz in some embodiments), and/or radio waves in the 300 MHz to 300 GHz range, and/or millimeter waves in the 30 GHz to 300 GHz range. In some embodiments, the radar has a bandwidth of at least 1 GHz. The active reflected wave detector 206 may comprise antennas for both emitting waves and for receiving reflections of the emitted waves, and in some embodiment different antennas may be used for the emitting compared with the receiving.

As will be appreciated the active reflected wave detector 206 is an “active” detector in the sense of it relying on delivery of waves from an integrated source in order to receive reflections of the waves. The active reflected wave detector 206 is not limited to being a radar sensor, and in other embodiments alternative ranging detectors may be used, for example the active reflected wave detector 206 may be a LIDAR sensor, or a sonar sensor.

The active reflected wave detector 206 being a radar sensor is advantageous over other reflected wave technologies in that radar signals may transmit through some materials, e.g. wood or plastic, but not others—notably water which is important because humans are mostly water. This means that the radar can potentially “see” a person in the environment even if they are behind an object of a radar-transmissive material. Depending on the material, this may not be the case for sonar or lidar.

The CPU 202 comprises a fall detector 214 which comprises a state classifier 216. The state classifier 216 is configured to determine the fall status of a person in the environment based on measured wave reflection data. The fall detector 214 is configured to determine the condition of a person in the environment using the output of the state classifier 216.

In operation, the active reflected wave detector 206 performs one or more reflected wave measurements at a given moment of time, and over time these reflected wave measurements can be correlated by the CPU 202 with the presence of a person and/or a state of the person and/or a condition of the person.

In the context of the present disclosure, the fall status of the person determined by the state classifier 216 may be a characterization of the person based on a momentary assessment (e.g. whether the person is in a fall state or a non-fall state). For example, a classification based on their position (e.g. in a location in respect to the floor and in a configuration which are consistent or inconsistent with having fallen) and/or their kinematics (e.g. whether they have a velocity that is consistent or inconsistent with them having fallen, or having fallen possibly being immobile).

In the context of the present disclosure, the condition of the person determined by the fall detector 214 may comprise a determination of an aspect of the person's health or physical predicament, for example whether they are in a fall condition whereby they have fallen and are substantially immobile, such that they may not be able (physically and/or emotionally) to get to a phone to call for help. That is, determining that a person is in a “fall condition” refers herein to determining that they have actually fallen.

The condition of the person may in some contexts be synonymous with the status of the person. For example, by determining that the person is in a standing state, it may be concluded by the fall detector 214 that the person is not currently in a fall condition, whereby they are on the floor and potentially unable to seek help.

It is possible to detect a fall based on a single detection of a fall state (e.g. that the person is in a position consistent with having fallen), but doing so has a relatively high risk of false alarms. Thus, in some embodiments the determination that a person is in a “fall condition” performed by the fall detector 214 involves an assessment of the person's fall status over time, such as in the order of 30-60 seconds, whereby multiple time separated determinations of the person having a fall status is needed in order to conclude there is a fall condition. For example, a person may be classified as being in a fall state by the state classifier 216 and then after a predetermined amount of time the fall status of the person is then reclassified by the state classifier 216 to see if the person is still in the same position, and if so, the fall detector 214 determines that there is a person in a fall condition (because they have been in a fall position for some amount of time deemed to indicate they may need help). Power can be advantageously conserved energy by switching the active reflected wave detector 206 to a lower power state (e.g. off or asleep) between the reflected wave measurements performed by the active reflected wave detector 206.

In some embodiments, the CPU 202 is configured to control the camera 210 to capture an image (represented by image data) of the environment. The camera 210 is preferably a visible light camera in that it senses visible light. Alternatively, the camera 210 senses infrared light. One example of a camera which senses infrared light is a night vision camera which operates in the near infrared (e.g. wavelengths in the range 0.7-1.4 μm) which requires infrared illumination e.g. using infrared LED(s) which is not visible to an intruder. Another example of a camera which senses infrared light is a thermal imaging camera which is passive in that it does not require an illuminator, but rather, senses light in a wavelength range (e.g. a range comprising 7 to 15 μm, or 7 to 11 μm) that includes wavelengths corresponding to blackbody radiation from a living person (around 9.5 μm). The camera 208 may be capable of detecting both visible light and, for night vision, near infrared light. The CPU 202 may comprise an image processing module for processing image data captured by the camera 210.

The device 102 may comprise a communications interface 214 for communication of data to and from the device 102. For example, the device 102 may communicate with a remote device via the communications interface 214. This enables a fall detection alert message to be sent from the device 102 to a remote device (not shown in FIGS. 1 and 1b), which may be via a wireless connection. This remote device may for example be a mobile computing device (e.g. a tablet or smartphone) associated with a carer or relative. Alternatively the remote device may be a computing device in a remote location (e.g. a personal computer in a monitoring station). Alternatively the remote device may be a control hub in the environment 100 (e.g. a wall or table mounted control hub). The control hub may be a control hub of a system that may be monitoring system and/or may be a home automation system. The notification to the control hub is in some embodiments via wireless personal area network, e.g. a low-rate wireless personal area network.

Additionally or alternatively, the device 102 may communicate, via the communications interface 214, with one or more of the activity sensor(s) 212, the active reflected wave detector 206, and the camera 210 in embodiments in which such components are not housed in the housing 200 of the device 102.

The device 102 may comprise an output device 208 to output a fall detection alert. For example, the CPU 202 may control a visual output device (e.g. a light or a display) on device 102 to output a visual alert of the fall detection. Alternatively or additionally, the CPU 202 may control an audible output device (e.g. a speaker) on device 102 to output an audible alert of the fall detection.

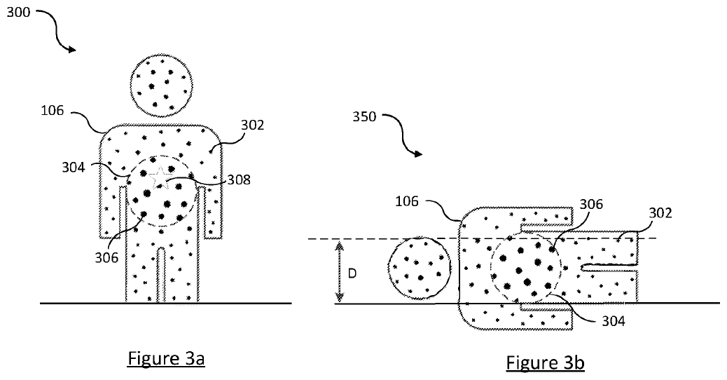

FIG. 3a illustrates a free-standing human body 106 with indications of reflective wave reflections therefrom in accordance with embodiments.

For each reflected wave measurement, for a specific time in a series of time-spaced reflective wave measurements, the reflective wave measurement may include a set of one or more measurement points that make up a “point cloud”. Each point 302 in the point cloud may be defined by a 3-dimensional spatial position from which a reflection was received, and defining a peak reflection value, and a doppler value from that spatial position. Thus, a measurement received from a reflective object may be defined by a single point, or a cluster of points from different positions on the object, depending on its size.

In some embodiments, such as in the examples described herein, the point cloud represents only reflections from moving points of reflection, for example based on reflections from a moving target. That is, the measurement points that make up the point cloud represent reflections from respective moving reflection points in the environment. This may be achieved for example by the active reflected wave detector 206 using moving target indication (MTI). Thus, in these embodiments there must be a moving object in order for there to be reflected wave measurements from the active reflected wave detector (i.e. measured wave reflection data), other than noise. The minimum velocity required for a point of reflection to be represented in the point cloud is less for lower frame rates. Alternatively, the CPU 202 receives a point cloud from the active reflected wave detector 206 for each frame, where the point cloud has not had pre-filtering out of reflections from moving points. Preferably for such embodiments, the CPU 202 filters the received point cloud to remove points having Doppler frequencies below a threshold to thereby obtain a point cloud representing reflections only from moving reflection points. In both of these implementations, the CPU 202 accrues measured wave reflection data which corresponds to point clouds for each frame whereby each point cloud represents reflections only from moving reflection points in the environment.

In other embodiments, no moving target indication (or any filtering) is used. In these implementations, the CPU 202 accrues measured wave reflection data which corresponds to point clouds for each frame whereby each point cloud can represent reflections from both static and moving reflection points in the environment. Even without removal of measurement points representing reflections from static objects the lower frame rate can still detect slower movements than at the higher frame rate.

FIG. 3a illustrates a map of reflections. The size of the point represents the intensity (magnitude) of energy level of the radar reflections (see larger point 306). Different parts or portions of the body reflect the emitted signal (e.g. radar) differently. For example, generally, reflections from areas of the torso 304 are stronger than reflections from the limbs. Each point represents coordinates within a bounding shape for each portion of the body. Each portion can be separately considered and have separate boundaries, e.g. the torso and the head may be designated as different portions. The point cloud can be used as the basis for a calculation of a reference parameter or set of parameters which can be stored instead of or in conjunction with the point cloud data for a reference object (human) for comparison with a parameter or set of parameters derived or calculated from a point cloud for radar detections from an object (human).

When a cluster of measurement points are received from an object in the environment 100, a location of a particular part/point on the object or a portion of the object, e.g. its centre, may be determined by the CPU 202 from the cluster of measurement point positions having regard to the intensity or magnitude of the reflections (e.g. a centre location comprising an average of the locations of the reflections weighted by their intensity or magnitude). As illustrated in FIG. 3a, the reference body has a point cloud from which its centre has been calculated and represented by the location 308, represented by the star shape. In this embodiment, the torso 304 of the body is separately identified from the body and the centre of that portion of the body is indicated. In alternative embodiments, the body can be treated as a whole or a centre can be determined for each of more than one body part e.g. the torso and the head, for separate comparisons with centres of corresponding portions of a scanned body.

In one or more embodiments, the object's centre or portion's centre is in some embodiments a weighted centre of the measurement points. The locations may be weighted according to an Radar Cross Section (RCS) estimate of each measurement point, where for each measurement point the RCS estimate may be calculated as a constant (which may be determined empirically for the reflected wave detector 206) multiplied by the signal to noise ratio for the measurement divided by R⁴, where R is the distance from the reflected wave detector 206 antenna configuration to the position corresponding to the measurement point. In other embodiments, the RCS may be calculated as a constant multiplied by the signal for the measurement divided by R⁴.

This may be the case, for example, if the noise is constant or may be treated as though it were constant. Regardless, the received radar reflections in the exemplary embodiments described herein may be considered as an intensity value, such as an absolute value of the amplitude of a received radar signal.

In any case, the weighted centre, WC, of the measurement points for an object may be calculated for each dimension as:

$$W C = \frac{1}{{\sum }_{n = 1}^{N} {W}_{n}} {\sum }_{n = 1}^{N} ( {W}_{n} {P}_{n} )$$

Where:
N is the number of measurement points for the object;
Wₙ is the RCS estimate for the n^{th} measurement point; and
Pₙ is the location (e.g. its coordinate) for the n^{th} measurement point in that dimension.

When determining how close an object is to the active reflected wave detector 206, the weighted centre, WC, of the measurement points for the object may optionally be used. In other words, the distance to the object may be calculated at the radial distance from the active reflected wave detector 206 to the position of the determined weighted centre of the of measurements corresponding to the object.

In some embodiments, the CPU 202 is configured to process measured wave reflections from the environment that are measured by the active reflected wave detector 206 to detect whether a person is in the environment and, if a person is detected, classify a state of the person in the environment.

As will be described in more detail below, this need not be a two-step process i.e. of looking for a person and then classifying them. For example, the CPU 202 may take the output of the active reflected wave detector 206 and do a classification, wherein one of the outputs of the classification is that there is no person, or in other embodiments it may only conclude that there is no person if it fails to perform a classification of a person's status.

When classifying the state of a person, the CPU 202 may perform a determination that the person is in a fall position (i.e. a position that is consistent with them haven fallen) or a non-fall position (indicative that they are, at least temporarily, in a safe state). In embodiments of the present disclosure the determination that the person is in a fall position is used as an indicator that the person may be in need of help. Being in a position which is consistent with the person having fallen does not necessarily mean they have fallen, or have fallen such that they need help. For example, they may be on the floor for other reasons, or they may have had a minor fall from which they can quickly recover. However, if they remain in a fall position for sufficient time it may be concluded that they are sufficiently likely to have fallen to be classified as being in a fall condition, and the device 102 may therefore take appropriate action accordingly, e.g. by sending a notification to a remote device.

In some embodiments, the classification performed by the CPU 202 may provide further detail on the non-fall condition for example, the CPU 202 may be able to classify the person as being in a state from one or more of a free-standing state (e.g. they are walking); a safe supported state which may be a reclined safe supported state whereby they are likely to be safely resting (e.g. a state in which they are in an elevated lying down position, or in some embodiments this may additionally encompass being in a sitting position on an item of furniture); and a standing safe supported state (e.g. they are standing and leaning on a wall). In other embodiments the non-fall states may be grouped differently. For example, the non-fall states may include a stationary non-floor position (encompassing both a reclined safe supported state and a standing stationary state whether supported or not in the standing state) and an ambulatory state. The CPU 202 may be able to classify the person as crawling, which may be regarded as a fall state or a non-fall state (given that if the person has fallen the person is still able to move so may be regarded as less critical) dependent on how the CPU 202 is configured.

The classification may be performed by the CPU 202 by looking at a set of sequential frames over a period of time and classifying the state of the person as being in a fall position based on the person's fall/non-fall positions for the respective frames. Multiple frames (e.g. 10 frames) may be used to determine whether there are more fall or non-fall results to improve the accuracy of the determination (the result which occurs more is the selected result).

### Using Thresholds

In some embodiments, in order to detect and classify the state of a person the processes the measured wave reflections by determining one or more parameters associated with the measured wave reflections and then comparing the parameter(s) to one or more thresholds to detect and classify the state of a person.

The person may be tracked using a tracking module of the state classifier 216. The tracking module can use any known tracking algorithm. For example, the active reflected wave detector 206 may generate a plurality of detection measurements (e.g. up to 100 measurements, or in other embodiments hundreds of measurements) for a given frame. Each measurement can be taken a defined time interval apart such as 0.5, 1, 2 or 5 seconds apart. Each detection measurement may include a plurality of parameters in response to a received reflective wave signal above a given threshold. The parameters for each measurement may for example include an x and y coordinate (and z coordinate for a 3D active reflected wave detector 206), a peak reflection value, and a doppler value corresponding to the source of the received radar signal. The data can then be processed using a clustering algorithm to group the measurements into one or more measurement clusters corresponding to a respective one or more targets. An association block may then associate a given cluster with a given previously measured target. A Kalman filter of the tracking module may then be used to determine the next position of the target based on the corresponding cluster of measurements and the prediction of the next position based on the previous position and other information e.g. the previous velocity.

From the reflected wave measurements an RCS of an object represented by a cluster of measurement points can be estimated by summing the RCS estimates of the each of the measurement points in the cluster. This RCS estimate may be used to classify the target as a human target if the RCS is within a particular range potentially relevant to humans for the frequency of the signal emitted by the active reflected wave detector 206, as the RCS of a target is frequency dependent. Taking a 77 GHz radar signal as an example, from empirical measurements, the RCS (which is frequency dependent) of an average human may be taken to be in the order of 0.5 m², or more specifically in a range between 0.1 and 0.7 m², with the value in this range for a specific person depending on the person and their orientation with respect to the radar. The RCS of human in the 57-64 GHz spectrum is similar to the 77 GHz RCS—i.e. 0.1 and 0.7 m².

The tracking module may output values of location, velocity and/or RCS for each target, and in some embodiments also outputs acceleration and a measure of a quality of the target measurement, the latter of which is essentially to act as a noise filter. The values of position (location) and velocity (and acceleration, if used) may be provided in 2 or 3 dimensions (e.g. cartesian or polar dimensions), depending on the embodiment.

The Kalman filter tracks a target object between frames and therefore multiple frames of reflection measurement data can be used to determine a person's velocity. Three or more frames (e.g. 3-5 frames) may be required in order to determine that there is movement exceeding a movement threshold. The frames may be taken at a rate of 2 Hz, for example.

In order to classify the state of the person in the environment, the state classifier 216 may determine a height metric associated with at least one measurement of a reflection from the person conveyed in the output of the active reflected wave detector 206 and compare the height metric to at least one threshold.

The height metric may be a height of a weighted centre of the measurement points of a body or part thereof (where each measurement is weighted by the RCS estimation), and the state classifier 216 may compare this height metric to a threshold distance, D, from the floor (e.g. 30 cm).

The height metric used to classify the state of the person is not limited to being a height of a weighted centre of the measurement points of the person's body or part thereof. In another example, the height metric may be a maximum height of all of the height measurements associated with the person's body or part thereof. In another example, the height metric may be an average height (e.g. median z value) of all of the height measurements of the person's body or part thereof. In the case of using a weighted centre or average height, the “part thereof” may beneficially be a part of the body that is above the person's legs to more confidently distinguish between fall and non-fall positions.

If the height metric (e.g. weighted centre, average height and/or maximum height) is within (less than) the threshold distance, D, from the floor (e.g. 30 cm), the state classifier 216 may determine that the person in the environment is in a fall position.

In order to classify the state of the person in the environment, the state classifier 216 may determine a velocity associated with the person using the measurements of reflections that are conveyed in the output of the active reflected wave detector 206 and compare the velocity to a velocity threshold. The tracking module referred to above may output a value of velocity for the target (person in the environment). For example, the velocity may assist in classifying whether a human is present in the environment. For example, it may be concluded that no human is present if there is no detected object having a velocity within a predefined range and or having certain dynamic qualities that are characteristic of a human. The comparison between the detected velocity associated with the person and the velocity threshold can also assist with narrowing the classification down to a specific state. For example if the detected velocity associated with the person is not greater than the velocity threshold the state classifier 216 may determine that the person is not moving and is in a fall state.

In order to classify the state of the person in the environment, the state classifier 216 may determine a spatial distribution, e.g. a variance or standard deviation, of the measurements of reflections that are conveyed in the output of the active reflected wave detector 206 and compare the spatial distribution to a threshold. This may include determining a horizontal spatial distribution of the measurements of reflections that are conveyed in the output of the active reflected wave detector 206 and comparing the horizontal spatial distribution to a horizontal spatial distribution threshold. Alternatively or additionally, this may include determining a vertical spatial distribution of the measurements of reflections that are conveyed in the output of the active reflected wave detector 206 and comparing the vertical spatial distribution to a vertical spatial distribution threshold.

The comparison between the spatial distribution(s) to a threshold can assist with narrowing the classification down to a specific state. For example, if the vertical spatial distribution is less than the vertical spatial distribution threshold (low z variance) and/or the horizontal spatial distribution is greater than the horizontal spatial distribution threshold (high x-y plane variance), then the state classifier 216 can determine that the person is in a fall state. Alternatively the ratio of the horizontal spatial distribution to vertical spatial distribution may be compared with a threshold. Such a ratio being above a threshold that has a value greater than 1 may be taken to indicate that the person is in a fall state.

### Using a Classifier Model

In other embodiments, in order to detect and classify the state of a person, rather than the state classifier 216 determining one or more parameters associated with the measured wave reflections and then comparing the parameter(s) to one or more thresholds, the state classifier 216 may supply the determined parameters as inputs into a trained classifier model.

The trained classifier model may be trained using one or more training data sets which include reflective wave measurements and a corresponding definition of which output state the reflective wave measurements correspond to. The trained classifier model may be trained based on reflections of single or multiple occupancy environments.

The received parameters may include one or more of (i) a height metric associated with at least one reflection; (ii) a velocity associated with the person using the measurements of reflections; and (iii) a spatial distribution characterization of the measurements (e.g. one or more of a horizontal spatial distribution (e.g. a variance or equivalently a standard deviation), a vertical spatial distribution and a ratio therebetween. Additionally, RCS estimates may be used to aid in assessing whether the object being classified is in fact a human. Analysis of the wave reflections to determine whether the object is likely to be human may be performed before or after the classification, but in other embodiments it may be performed as part of the classification. Thus, the classifier may additionally receive the following parameters: (iv) a sum of RCS estimates, and in some embodiments (v) a distribution (e.g., variance or equivalently standard deviation) of RCS estimates. For example, the received parameters may be: 1. an average height (e.g. median z value); 2. a standard deviation of RCS estimates; 3. A sum of RCS estimates; and 4. a standard deviation of height(z) values.

In these embodiments the trained classifier model uses the received parameters and the training data set(s) to classify the state of the person in the environment.

It will be appreciated that this can be implemented in various ways.

The trained classifier model may be used at operation time to determine a classification score, using a method known by the person skilled in the art. The score may for example provide an indication of a likelihood or level of confidence that the received parameters correspond to a particular classifier output state. A determination of a particular classification (e.g. a fall position) may for example be based on whether a classification confidence score is greater than a threshold then the person is determined to be in that state. For example, the CPU 202 may determine that the person is in a fall state if the output of the classifier determines that there is more than a 60% likelihood (or some other predefined likelihood threshold, which may optionally be greater than 50%, or even less than 50% to be conservative/cautious) of the person being in a fall position. However, in some embodiments to ameliorate the risk of a false positive fall detection, the threshold confidence for determine that the person is in a fall state is in the range of 85-90%, e.g. 88%. In other words, if there is a confidence of being in a fall state that is greater than the threshold confidence it may concluded that the person is in a fall state. Likewise, if there is a confidence of being in a fall state that is less than the threshold confidence (88% in this example) then it may be concluded that the person is in a non-fall state.

It will be appreciated that it may not be necessary for the classifier model to be trained with a data set associated with a particular classifier state in order for the classifier model to classify the person as being in the particular classifier state. Consider the simple example whereby the trained classifier model is configured to indicate that the person is in one of two states (i.e. in a fall state or a non-fall state), the trained classifier model may have been trained with a data set including reflective wave measurements corresponding to a person in a non-fall state, and based on a low correlation of the received parameters to the training data set corresponding to a person in a non-fall state, the trained classifier model may be configured to indicate that the person is in a fall state.

Furthermore, as noted above, there need not be a two-step process of looking for a person and then classifying them. A trained classifier model could be used that is trained of different data that is not necessarily limited to reflections from discreet objects or from objects already identified as potentially being human. For example, a classifier could be fed respective sets of training data for (i) a person is present and in a fall position; (ii) a person is present and in a non-fall position; and (iii) no person is present. The classifier may determine a classification of active reflective wave measurements based on which of the trained states it is most closely correlated with.

Any other method, known by the person skilled in the art, of training and using the classifier based on (i) the receiving parameters as exemplified above, and (i) the relevant output states may alternatively be used.

Regardless of how the state classifier 216 classifies the state of a person, if the fall detector 214 detects that a person in the environment has fallen, the fall detector 214 is configured to output a fall detection alert. The fall detector 214 may output the fall detection alert via the output device 208 (e.g. a visual and/or audible alert). Alternatively or additionally, the fall detector 214 may output the fall detection alert to a remote device via the interface 214.

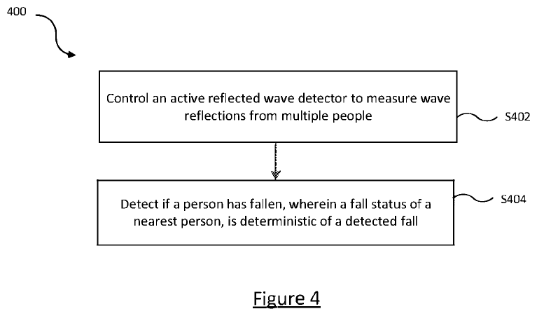

We now refer to FIG. 4 which illustrates a process 400 performed by the CPU 202 to detect if a person has fallen in an environment comprising multiple people in accordance with embodiments of the present disclosure.

At step S402, the CPU 202 controls the active reflected wave detector 206 to measure wave reflections from the environment comprising multiple people and receives measured wave reflection data that is obtained by the active reflected wave detector.

It should be noted that when the process 400 is started, the active reflected wave detector 206 may be in a deactivated state. In the deactivated state the active reflected wave detector 206 may be turned off. Alternatively, in the deactivated state the active reflected wave detector 206 may be turned on but in a lower power consumption operating mode whereby the active reflected wave detector 206 is not operable to perform reflected wave measurements.

In these implementations, the CPU 202 is configured to use an activity sensor 212 to monitor the activity in the environment 100, and if no activity is detected for a predetermined amount of time, then the CPU 202 activates the active reflected wave detector 206 so that it is in an activated state (e.g. a higher power consumption operating mode) and operable to measure wave reflections from the environment 100.

The active reflected wave detector 206 consumes more power in an activated state (i.e. when turned on and operational) than the activity sensor 212 in an activated state. Thus using a relatively low power consuming activity sensor (e.g. a motion detector such as a PIR detector) to determine whether there is activity (e.g. movement) in the environment 100, ensures that the active reflected wave detector 206 is only fully operational when activity is no longer detected (the person has stopped moving enough to be detected by the activity sensor 212 meaning that they may have fallen, or they can't be seen by the activity sensor).

At step S404, the fall detector 214 uses the output of the state classifier 216 to detect whether a person has fallen based on the measured wave reflection data. At step S404, a fall status of a nearest person in the environment to the active reflected wave detector is deterministic of a detected fall. Expressed another way, whether or not a nearest person in the environment is in a position consistent with having fallen is deterministic of a detected fall.

As a result, in embodiments of the present disclosure the fall detector 214 detects that a person has not fallen in the environment if the nearest person in the environment is in a non-fall state regardless of the fall status of a person that is not nearest to the active reflected wave detector 206 (referred to herein as a non-nearest person). Thus, even if a non-nearest person is in a fall state, the fall detector 214 will not detect that a person has fallen in the environment and will not issue a fall detection alert. This is because in embodiments of the present disclosure a fall status of a non-nearest person is not deterministic of a detected fall.

Furthermore, in embodiments of the present disclosure the fall detector 214 detects that a person has fallen in the environment if the nearest person in the environment is in a fall state regardless of the fall status of a non-nearest person. Thus the fall detector 214 detects that a person has fallen in the environment if. (i) the nearest person in the environment is in a fall state and a non-nearest person is in a non-fall state; or (ii) the nearest person in the environment is in a fall state and a non-nearest person is in a fall state.

Step S404 may be performed in a number of different ways.

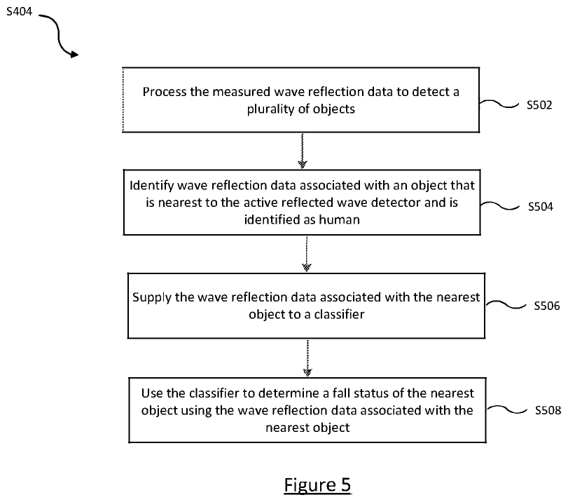

In one implementation, during step S404 the fall status of the nearest person to the active reflected wave detector 206 is determined by the state classifier 216. This implementation is illustrated in FIG. 5 which illustrates steps that may be performed during step S404.

As shown in FIG. 5, at step S502 the CPU 202 processes the measured wave reflection data to detect a plurality of objects. The plurality of detected objects will correspond to the different people in the environment and may also include one or more phantom objects. To perform step S502, the CPU 202 may process the measured wave reflection data using a clustering algorithm to group the measurements into measurement clusters corresponding to respective detected objects.

At step S504, the CPU 202 identifies wave reflection data associated with the object, of the plurality of detected objects, which is nearest to the active reflected wave detector 206 and is identified as potentially representing a human. For example, one or more parameters (e.g. an RCS estimate, velocity, etc.) derived or calculated from the wave reflection data associated with the nearest object can be used to identify the nearest object as potentially human. Optionally a Kalman filter may be used, e.g. as part of a tracking module, as part of the algorithm used to identify objects potentially representing human. It is appreciated that detected objects that may be identified as potentially human may include actual humans, phantom humans, or other objects (e.g. large pets) that could be confused as being human, depending on the human identification process. To minimise false results, the use of the device 102 may optionally be used in environments in which such other objects are not expected to be present.

At step S506, the CPU 202 supplies the wave reflection data associated with the nearest object to the state classifier 216.

Analysis of the wave reflection data associated with the nearest object to determine whether the object is likely to be human may be performed before classification performed at step S508, but in other embodiments it may be performed as part of the classification at step S508.

At step S508, the state classifier 216 determines the fall status of the nearest person to the active reflected wave detector 206 using the wave reflection data associated with the nearest object. The wave reflection data associated with the nearest object identified as human (i.e. at least potentially human, as discussed above) will correspond to the person in the environment that is nearest to the active reflected wave detector 206 and will not be a phantom object due to the fact that any phantom object will be as a result of wave reflections that have travelled on an indirect path from a person in the environment.

In this implementation, the state classifier 216 does not receive wave reflection data associated with identified objects that are not the nearest to the active reflected wave detector 206.

As noted above, the fall detector 214 may determine if a person has not fallen based on a single assessment of the nearest person's fall status at step S508. Additionally, or alternatively, in other embodiments, the fall detector 214 may determine if a person has fallen based on a single assessment of the nearest person's fall status at step 508.

Alternatively once the state classifier 216 has detected that the nearest person is in a fall state at step S508, after a predetermined amount of time the fall status of the person is then reclassified by the state classifier 216 (by performing the steps of S404 shown in FIG. 5) to see if the person is still in a fall state, and if so, the fall detector 214 determines that there is a person in a fall condition (because they have been in a fall position for some amount of time deemed to indicate they may need help).

As optional step, if the CPU 202 receives a signal indicative of activity in the environment from the activity sensor 212 during the predetermined amount of time, then the CPU 202 does not perform the second classification of the nearest person's fall status. Thus in these implementations, the CPU 202 only issues a fall detection alert if there is a time window having at its start and end, respective fall position determinations by the state classifier 26, and with no movement detected there between by a (separate and low power) activity sensor 212.

In some embodiments, at step S508, in order to detect and classify the fall status of the nearest person the state classifier 216 processes the wave reflection data associated with the nearest object to determine one or more parameters (referred to above) and then compares the parameter(s) to one or more thresholds (for example as described above) to detect and classify the state of the nearest person as being in a fall state or a non-fall state.

Alternatively, at step S508, the state classifier 216 may supply the determined parameters as inputs into a trained classifier model. The trained classifier model may be trained using one or more training data sets which include reflective wave measurements and a corresponding definition of which output state (fall status) the reflective wave measurements correspond to. For example the one or more training data sets may comprise (i) a non-fall labelled dataset comprising reflective wave measurements of one or more environments comprising a single person (i.e. only one person) and that single person is in a non-fall state (e.g. in a standing position, or elevated sitting, reclined, or lying position); and/or (ii) a fall labelled dataset comprising reflective wave measurements of one or more environments comprising a single person, and that single person is in a fall state. The data sets may further comprise: (iii) a non-fall labelled dataset comprising reflective wave measurements of one or more environments comprising multiple people, all of whom are in a non-fall state; and/or (iv) a fall labelled dataset comprising reflective wave measurements of one or more environments comprising multiple people, in which at least one of the multiple people is a person in a fall state, and the least one of the multiple people in a fall state comprises the nearest person to the active reflective wave detector. As will be appreciated these training data sets may also include phantom objects. Thus, in some embodiments, even in the case of single-person data sets, the data sets may in some embodiments be limited to include only the measurements corresponding to the nearest person.

In a similar manner, the CPU 202 may be further configured to detect if a person alone in an environment has fallen. That is, the CPU 202 may use the process described above to detect if a person has fallen in an environment comprising only the single person.

In these embodiments, CPU 202 controls the active reflected wave detector 206 to measure wave reflections from the environment comprising the single person and receives measured wave reflection data that is obtained by the active reflected wave detector.

The CPU 202 may then perform step S404 shown in FIG. 5 whereby the plurality of objects detected at step S502 will correspond to the person in the environment and one or more phantom objects. For example, with reference to FIGS. 1 and 1b, in the case of a single person being in the environment, the actual person would the person indicated by reference numeral 106, whereas the person indicated by reference numeral 108 would be a phantom object or, in other words, an echo of person 106.

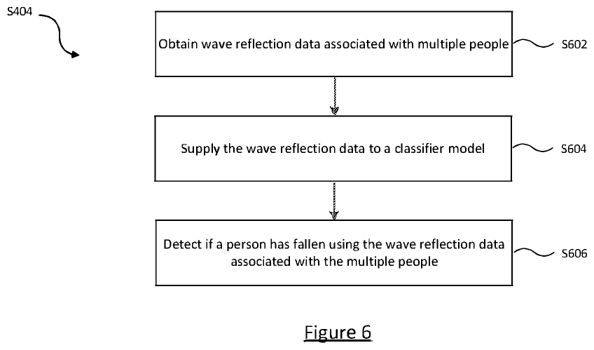

We now refer to an alternative way that step S404 may be performed with reference to FIG. 6. In this example implementation, the outcome of the fall detection is dependent on the fall position of the nearest person to the active reflected wave detector 206, but the nearest person is not identified by the device 102 and there is no identification of which measured reflected wave data corresponds specifically to that person and not to other objects (real or phantom) represented in the measured reflected wave data.

At step S602, the CPU 202 obtains measured wave reflection data from wave reflections from the multiple people in the environment.

The obtained measured wave reflection data comprises wave reflection data associated with the nearest person to the active reflected wave detector 206 and also wave reflection data associated with people in the environment that are not the nearest to the active reflected wave detector 206.

It will be appreciated that the measured wave reflection data obtained by the CPU 202 at step S602 may also comprise wave reflections that have travelled on an indirect path from a person in the environment thereby giving rise to one or more phantom objects.

At step S604, the CPU 202 processes the obtained measured wave reflection data that is associated with the multiple people in the environment to determine one or more parameters (referred to above) and the CPU 202 may supply the determined parameters as inputs into a trained classifier model. The trained classifier model may be trained using one or more training data sets associated with a multi-occupancy environment (i.e. with multiple people present) which include reflective wave measurements and are labelled as being representative that a person has fallen in the environment or being representative that a person has not fallen in the environment.

For example the one or more training data sets may comprise at least one of (i) a first wave reflection data training set comprising wave reflection data of a nearest person to an active reflected wave detector being in a non-fall state, the first wave reflection data training set labelled as being representative that a person has not fallen in the environment; (ii) a second wave reflection data training set comprising wave reflection data of a nearest person to an active reflected wave detector being in a fall state and wave reflection data of a non-nearest person to an active reflected wave detector being in a non-fall state, the second wave reflection data training set labelled as being representative that a person has fallen in the environment; and (iii) a third wave reflection data training set comprising wave reflection data of a nearest person to an active reflected wave detector being in a fall state and wave reflection data of a non-nearest person to an active reflected wave detector being in a fall state, the third wave reflection data training set labelled as being representative that a person has fallen in the environment.

The first wave reflection data training set may comprise: (i) wave reflection data of a nearest person to an active reflected wave detector being in a non-fall state and wave reflection data of a non-nearest person to an active reflected wave detector being in a non-fall state, being labelled as being representative that a person has not fallen in the environment; and (ii) wave reflection data of a nearest person to an active reflected wave detector being in a non-fall state and wave reflection data of a non-nearest person to an active reflected wave detector being in a fall state, labelled as being representative that a person has not fallen in the environment.

Thus, in the example implementation shown in FIG. 6 at step S606 the CPU 202 is able to detect if a person has fallen in the environment if a classification score (that the received parameters correspond to a person having fallen in the environment) is greater than a predetermined threshold.

In the example implementation shown in FIG. 6, the CPU 202 is able to detect if a person has fallen in the environment without it being necessary to specifically identify wave reflection data associated with the person who is nearest to the active reflected wave detector 206.

In a similar manner to FIG. 6 described above, the CPU 202 may also detect if a person alone in an environment has fallen. That is, the CPU 202 is also able to detect if a person has fallen in an environment comprising only the single person, without any identification of which measured reflected wave data corresponds specifically to that person and not to other objects (e.g. phantom objects) represented in the measured reflected wave data.

In these embodiments, CPU 202 controls the active reflected wave detector 206 to measure wave reflections from the environment comprising the single person and receives measured wave reflection data that is obtained by the active reflected wave detector. In these examples, the measured wave reflection data obtained at step S602 will comprise wave reflection data associated with the single person and may also include wave reflection data associated with one or phantom objects.

The example implementations shown in FIGS. 5 and 6 may be performed individually or in combination. For example the first example implementation shown in FIGS. 4 and 5 could give a determination on whether a person is in a fall state, and the second example implementation shown in FIGS. 4 and 6 could run in parallel and also give a determination on whether a person is in a fall state. To make a final determination on whether or not to detect a fall, CPU 202 could optionally then combine or weight the respective determinations (fall/non-fall) according to their corresponding determination confidences. For example, to make the final determination on whether or not a person is in a fall state, the CPU may take an average of the classification confidence score of a person being in a fall state determined using the first example implementation, and the classification confidence score of a person being in a fall state determined using the second example implementation. In another example, to make the final determination on whether or not a person is in a fall state, the CPU may use the maximum value of (i) the classification confidence score of a person being in a fall state determined using the first example implementation, and (ii) the classification confidence score of a person being in a fall state determined using the second example implementation, i.e. whichever classification confidence score is highest. In yet another example, to make the final determination on whether or not a person is in a fall state, the CPU may use the minimum value of (i) the classification confidence score of a person being in a fall state determined using the first example implementation, and (ii) the classification confidence score of a person being in a fall state determined using the second example implementation, i.e. whichever classification confidence score is lowest.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer implemented method for detecting if a person has fallen in an environment comprising at least one person, the method comprising:
controlling an active reflected wave detector to measure wave reflections from environment to receive measured wave reflection data that is obtained by the active reflected wave detector;
detecting a plurality of objects from the measured wave reflection data that is obtained by the active reflected wave detector;
identifying wave reflection data associated with an object, of the plurality of objects, that is nearest to the active reflected wave detector and is identified as being human; and
detecting if a person has fallen in the environment by determining a fall status of a nearest person in the environment using the wave reflection data associated with the object in the environment that is nearest to the active reflected wave detector.

2. (canceled)

3. (canceled)

4. (canceled)

5. The computer implemented method of claim 1, wherein if the fall status of the nearest person is that they are in a non-fall state, the method comprising detecting that a person has not fallen in the environment.

6. (canceled)

7. The computer implemented method of claim 1, wherein the detecting if a person has fallen in the environment comprises disregarding wave reflection data associated with any objects that are not nearest to the active reflected wave detector.

8. The computer implemented method of claim 1, wherein the plurality of objects comprises the multiple people.

9. The computer implemented method of claim 8, wherein different people correspond to different objects of the plurality of objects.

10. The computer implemented method of claim 8, wherein the plurality of objects further comprise one or more phantom objects.

11. The computer implemented method of claim 1, the method comprising supplying the wave reflection data, that is associated with the object in the environment that is nearest to the active reflected wave detector, to a classifier that has been trained with training data, the classifier configured so that the detecting if a person has fallen in the environment is based on the training data and the wave reflection data that is associated with the object in the environment that is nearest to the active reflected wave detector.

12. (canceled)

13. (canceled)

14. (canceled)

15. (canceled)

16. The computer implemented method of claim 1, wherein the fall status of the nearest person represents whether the nearest person is in a position representative of them having fallen, wherein the nearest person is in a fall state if the nearest person is in a position representative of them having fallen, and is in a non-fall state if the nearest person is not in a position representative of them having fallen.

17. The computer implemented method of claim 1, wherein said controlling the active reflected wave detector to measure wave reflections from the environment is performed in response to detecting motion in the environment based on receiving motion detection data from a motion detector.

18. The computer implemented method of claim 17, wherein said controlling the active reflected wave detector to measure wave reflections from the environment is performed upon expiry of a time window that commences in response to the motion sensor detecting motion of a person.

19. (canceled)

20. A device for detecting if a person has fallen in an environment comprising at least one person, the device comprising:
a processor, wherein the processor is configured to:
control an active reflected wave detector to measure wave reflections from the environment to receive measured wave reflection data that is obtained by the active reflected wave detector;
detect a plurality of objects from the measured wave reflection data that is obtained by the active reflected wave detector;
identify wave reflection data associated with an object, of the plurality of objects, that is nearest to the active reflected wave detector and is identified as being human; and
detect if a person has fallen in the environment by determining a fall status using the wave reflection data associated with the object in the environment that is nearest to the active reflected wave detector.

21. (canceled)

22. A device according to claim 20, wherein the device further comprises the active reflected wave detector.

23. (canceled)

24. A device according to claim 20, wherein the processor is configured to disregard wave reflection data associated with any objects that are not nearest to the active reflected wave detector in said detection of a person has fallen in the environment.

25. A non-transitory computer-readable storage medium comprising instructions for detecting if a person has fallen in an environment comprising at least one person, the instructions when executed by a processor cause the processor to:
control an active reflected wave detector to measure wave reflections from the environment to receive measured wave reflection data that is obtained by the active reflected wave detector;
detect a plurality of objects from the measured wave reflection data that is obtained by the active reflected wave detector;
identify wave reflection data associated with an object, of the plurality of objects, that is nearest to the active reflected wave detector and is identified as being human, and detect if a person has fallen in the environment by determining the fall status using the wave reflection data associated with the object in the environment that is nearest to the active reflected wave detector.

26. A system for detecting if a person has fallen in an environment comprising at least one person, the system comprising at least one processor configured to perform a method of:
controlling an active reflected wave detector to measure wave reflections from the environment to receive measured wave reflection data that is obtained by the active reflected wave detector;
detecting a plurality of objects from the measured wave reflection data that is obtained by the active reflected wave detector;
identifying wave reflection data associated with an object, of the plurality of objects, that is nearest to the active reflected wave detector and is identified as being human; and
detecting if a person has fallen in the environment by determining a fall status using the wave reflection data associated with the object in the environment that is nearest to the active reflected wave detector.

27. The system of claim 26, further comprising the active reflected wave detector.

28. The system of claim 26, wherein the system comprises multiple processors to perform the method, the multiple processors distributed across two or more devices.

29. The computer implemented method of claim 1, wherein the plurality of objects comprise a single person in the environment and one or more phantom objects.
